(19) United States
(12) Patent Application Publication
(10) Pub. No.: US 2019/0232032 A1
(43) Pub. Date: Aug. 1, 2019

(54) Applicator for Providing Localized Relief and Treatment for Trauma, Injury, Post-Operative Care, and Cosmetic Use

(71) Applicant: EMA Associates, LLC, Oldsmar, FL (US)

(72) Inventor: Elaine Hewitt, Oldsmar, FL (US)

(21) Appl. No.: 15/881,264

(22) Filed: Jan. 26, 2018

(51) Int. Cl. A61M 35/00; A45D 40/26

(52) U.S. Cl. CPC A61M 35/003; A45D 40/26

(57) ABSTRACT

An applicator for providing localized relief and treatment to sensitive areas of the face, head, or body as a result of injury, trauma, operation, or cosmetic use. The applicator has an inner layer and an outer layer. The inner layer is saturated with a liquid to provide treatment or relief to the target area. The outer layer is at least semi-permeable so that when the applicator contacts the target area, the treatment liquid will seep out of the inner layer and pass through the outer layer onto to the skin or wound at the target area.

### FIELD

This invention relates to the field of cosmetic and post-operative relief and treatments, and more particularly to an applicator for relieving, healing, or treating swelling, irritation, infection, or pain in localized areas on the face, head, or body.

### BACKGROUND

The human body's healing and immune responses have certain side effects that can be undesirable. For example, when the body is healing or fighting infection, the localized site may become inflamed, swollen, or irritable during the healing process. Additionally, cuts, scrapes, scars, incisions, or other open wounds are susceptible to infection.

One specific example of this occurs after surgery. Doctors stitch incisions with sutures that remain in while the skin heals. This area can become itchy, swollen, or inflamed. These problems are especially bothersome when the surgery site is in a sensitive area such as the face.

Another example that most people have experienced in their daily lives is the swollenness that occurs under the eyes as a result of lack of sleep, crying, or allergies—commonly referred to as “bags” under the eyes.

Other sources of skin irritation and inflammation include bee stings, insect bites, and pimples.

What is needed is a device that provides localized relief and treatment to sensitive areas caused by injury, trauma, or surgical procedures.

### SUMMARY

The applicator allows a user to apply treatment to a localized area on the face, head, or body thereby providing relief for scars, bruises, scrapes, cuts, bug bites, pimples, or other skin injuries.

The applicator has two main portions—a handle and a head. The handle is made of a semi-rigid material such as wood, plastic, metal, or polymer.

The handle insulates the user from the head of the applicator. Because the user holds the applicator handle, she avoids cold fingers, melting ice, and touching salves or ointments.

Additionally, the length of the handle varies to allow the user to treat hard to reach areas on the body. If the area to be treated is on a face or hand a shorter handle is sufficient. If the area to be treated is hard to reach, such as the back, a longer handle is required.

Now turning to a discussion of the head of the applicator.

In one embodiment, the inner layer is a semi-permeable and at least partially absorbent material or fabric. Suitable fabrics include cotton, linen, wool, hemp, or other similar materials. The absorbent material will absorb and store treatment liquid that can then be applied to the skin by touching the head to the desired area.

In a second embodiment, the inner layer is a gel pouch. The gel retains heat and cold longer than pure water. The inner gel layer is preferred when applying a hot or cold compress to a sensitive area. Suitable gels include ethylene glycol, diethylene glycol, or similar materials that provide both hot and cold temperature retention.

In order to minimize irritation, the outer layer is an irritation-resistant fabric. The irritation-resistant fabric is permeable to liquids and gentle on the skin. Suitable fabrics include cotton, microfiber, or other synthetic materials. Thus, when the head contacts the sensitive area, the heat, cold, or treatment liquid transfers from the inner layer, through the outer layer, and onto the skin.

In embodiments with the inner gel pouch, the outer layer may be a plastic or polymer. When applied to the skin, the plastic or polymer layer conducts the heat or cold temperatures from the inner gel layer to the skin.

Now turning to a discussion of a kit for use after a surgical procedure. A selection of pre-packaged applicators may be grouped and sold as a kit. Such a kit is then sold at the store or given to a patient by a doctor. A kit containing multiple applicators provides an easy way for post-operative patients to obtain relief at home.

After a surgical procedure, icing and heating are often used on the area to reduce swelling, pain, or irritation. Icing or heating small areas can be cumbersome with a large heat pad or a bag of ice. The large heat pad is too large for areas on the face and placing ice in a bag results in messes due to condensation or leakage. In addition to icing or heating, some skin conditions require anti-biotic or antiseptic treatments to prevent infection.

The applicators with the absorbent inner layer are individually wrapped in a tear away bag, zip-top bag, a plastic tube with a cap, or other suitable container. The head of each applicator is saturated with a liquid or mixture of liquids having various medicinal properties.

Some kits may include a mixture of both the absorbent inner layer models and gel pouch models. The user then has a range of options from which to choose for her desired relief.

Each applicator can then be stored in the refrigerator or freezer until it is time to use. Alternatively, the applicators may be stored at room temperature and heated when it is time to use.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates an applicator in use over the eye of a patient that has had eyelid surgery.

FIG. 2 illustrates the applicator in use on bags underneath the eyes.

FIG. 3 illustrates a second embodiment of the applicator having a longer handle for use on hard to reach body parts.

FIG. 4 illustrates the two primary parts of the applicator.

FIG. 5 illustrates a side view of an applicator.

FIG. 6 illustrates a peal-away view of the head of the applicator is shown with the inner layer being an absorbent fabric.

FIG. 7 illustrates a peal-away view of the head of the applicator is shown with the inner layer being a gel.

FIG. 8 illustrates a third embodiment of the applicator.

FIG. 9 illustrates a side view of the third embodiment of the applicator.

FIG. 10 illustrates an individually wrapped, pre-saturated applicator in a storage bag.

FIG. 11 illustrates a storage bag containing three pre-saturated applicators.

FIG. 12 illustrates a rack containing a plurality of individually packaged, pre-saturated applicators.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

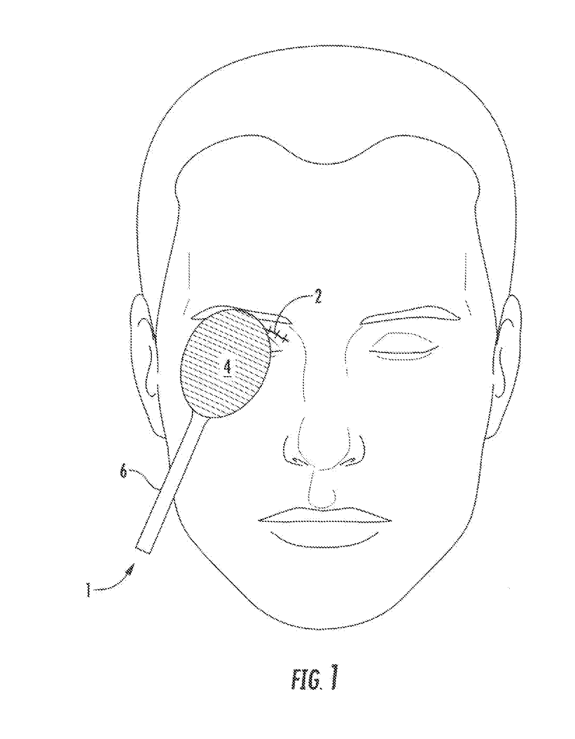

Referring to FIG. 1, a patient applying the applicator to a surgical scar on his eyelid is shown. The applicator 1 has a handle 6 and a head 4. The applicator 1 is held by the handle 6 so that a user may press or apply the head 4 to a bruise, scratch, scar, or other sensitive area 2.

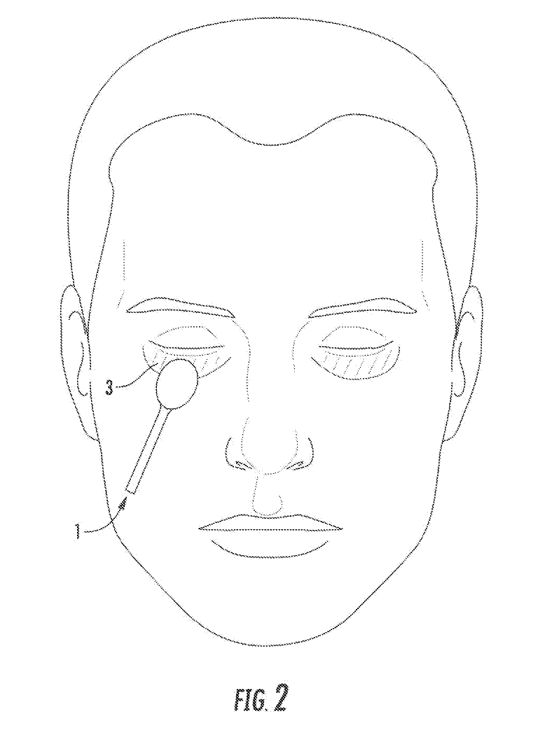

Referring to FIG. 2, a second intended use of the applicator is shown. Here, the applicator 1 is being applied to the underside of an eye 3 where bags formed due to lack of sleep.

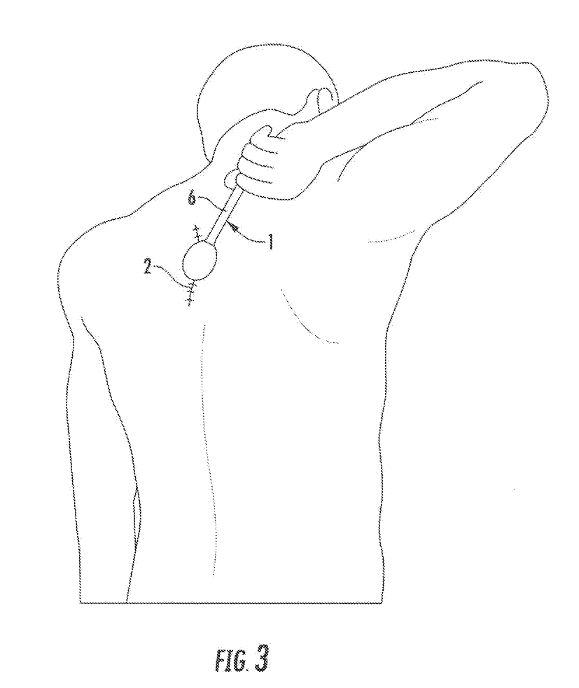

Referring to FIG. 3, another use of the applicator is shown. Here, the applicator 1 is being applied to a scar 2 between the shoulder blades.

In this embodiment of the applicator 1, the handle 6 will be longer than other embodiments to allow for easier application on difficult to reach areas.

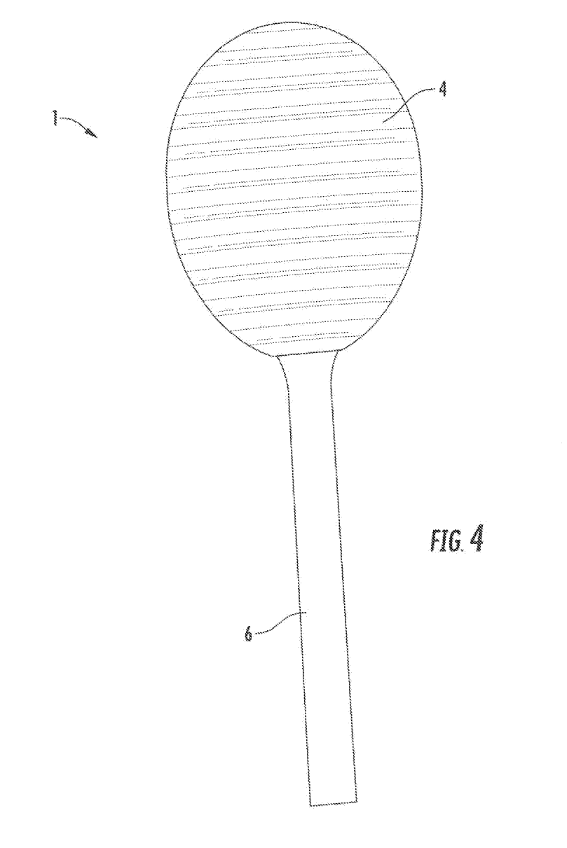

Referring to FIG. 4, a front view of an applicator is shown. The applicator 1 is comprised of a head 4 and a handle 6.

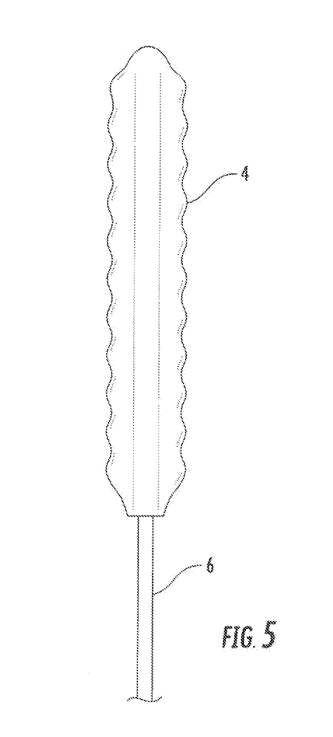

Referring to FIG. 5, a side view of an applicator is shown.

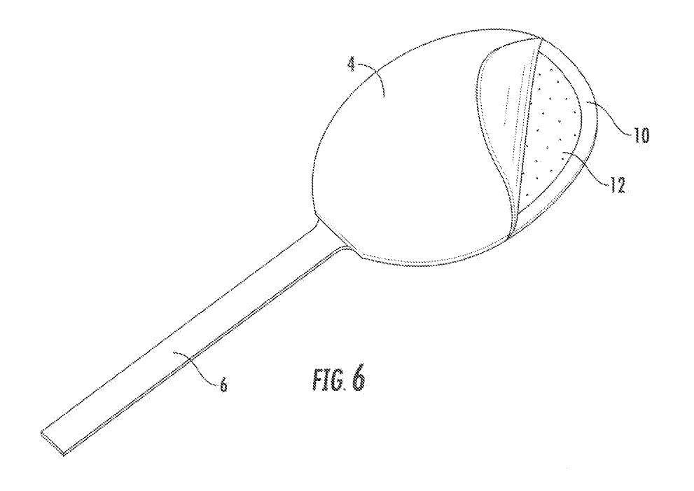

Referring to FIG. 6, a peal-away view of the head of an applicator is shown with the inner layer being an absorbent fabric.

In some embodiments, the inner layer 12 of the first embodiment is an absorbent fabric such as cotton, wool, linen, hemp, or foam. The inner layer 12 is saturated with distilled water, an antibiotic agent, an antiseptic agent, an anti-inflammatory agent, or a combination thereof.

The outer layer 10 is a soft, semi-permeable fabric such as cotton, microfiber, silk, or other suitable material.

When the head 4 of the applicator 1 contacts the sensitive area (e.g. a scar 2 or under the eye 3) the liquid stored by the absorbent inner layer 12 will leech through the semi-permeable outer layer and be delivered directly to sensitive area.

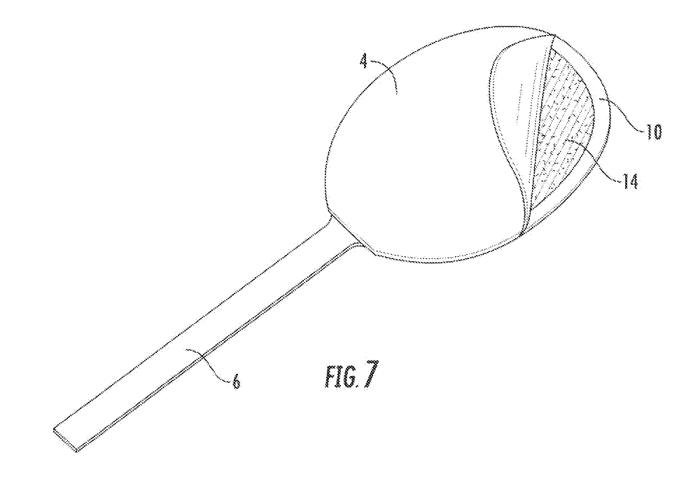

Referring now to FIG. 7, a second embodiment of the applicator is shown having an inner gel layer 14. In this embodiment, the inner gel layer 14 is ethylene glycol, diethylene glycol, or other suitable material that will retain heat without boiling, or retain cold without freezing.

In the second embodiment, the outer layer 10 is a non-irritating fabric such as cotton, microfiber, foam, or other suitable material.

An exemplary use of the second embodiment of the applicator 1 is to heat the applicator 1, in boiling water or a microwave, and apply the head 4 to a desired area.

Alternatively, the applicator 1 with an inner gel layer 14 may be stored in the freezer. The gel will not freeze, thereby resulting in a cold compress that retains flexibility to mold to the contours of a sensitive area.

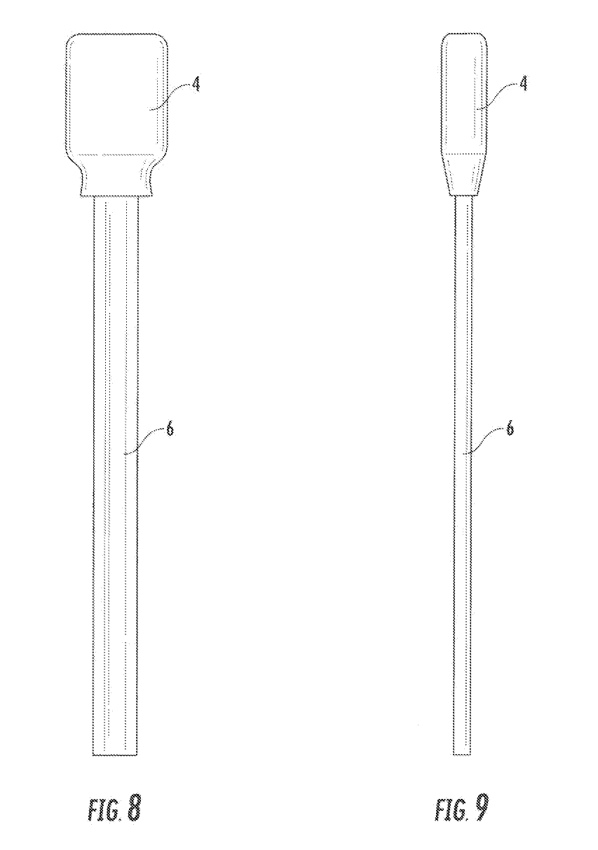

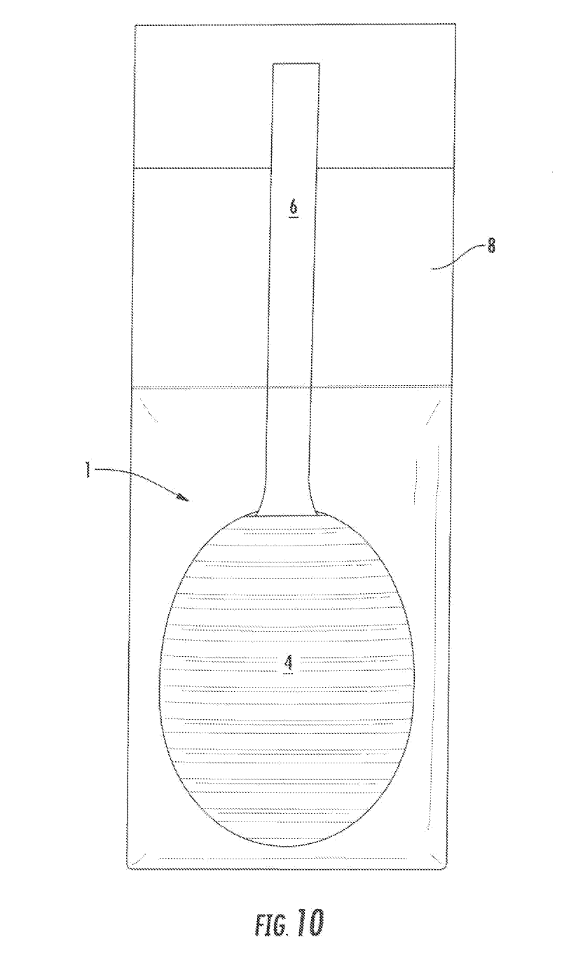

Referring to FIGS. 8 and 9, a third embodiment of the applicator is shown. This embodiment is suitable for treating under eye bags or swollenness because the head 4 of the third embodiment is smaller than in other embodiments which makes it easier to apply to a smaller body part or region.

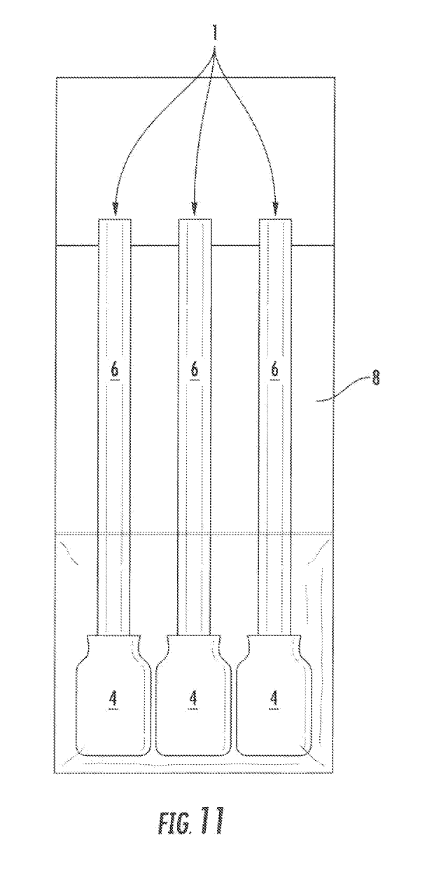

Referring to FIG. 10, an individually wrapped applicator is shown. For easy use, an applicator 1 may be pre-saturated and individually packaged into a storage bag 8 before being provided to patients or customers. Individually packaging the applicators 1 will prevent messes in the refrigerator, freezer, or microwave when cooling or heating the applicator 1 before use.

Additionally, a package containing multiple individually wrapped applicators 1 will have each head 4 saturated with a different liquid, solution, ointment, antibiotic, antiseptic, or sterile fluid.

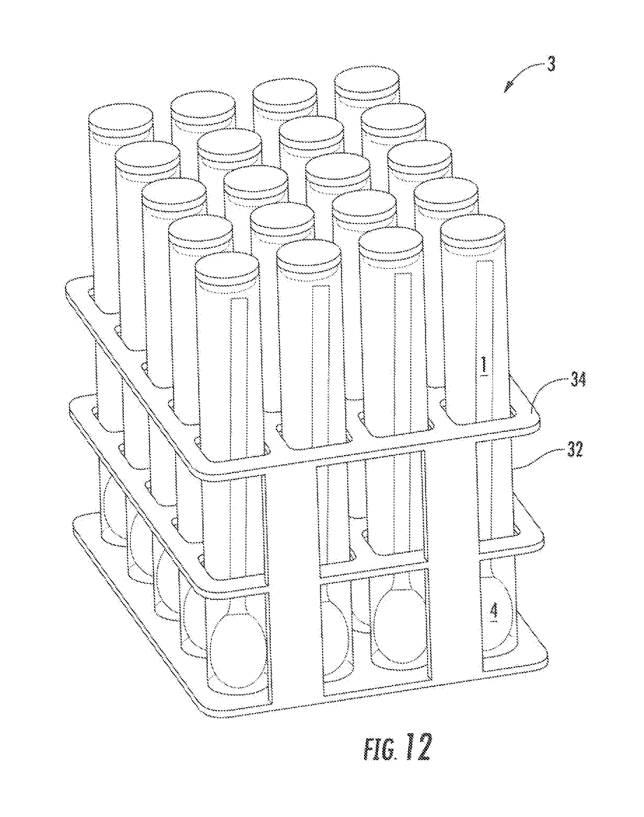

Referring to FIG. 11, a package with multiple applicators 1 in the same storage bag 8 is shown. Multiple applicators 1 may be stored in the same storage bag 8 if it is desirable to saturate all the heads 4 with the same treatment or relief liquid.

Referring to FIG. 12, an example of multiple individually wrapped applicators being provided in one package is shown. Each applicator 1 is individually stored in a storage tube 32 and a plurality of storage tubes 32 are placed into a rack 34. This single unit of multiple applicators is then provided to a customer, patient, doctor's office, or retailer. The benefit of this packaging is that each head 4 may be saturated with a different treatment or relief liquid.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. An applicator for cosmetic or medicinal use to provide treatment and relief to localized areas on a body, the applicator comprising:
a. a handle, the handle having a first end and a second end;
b. a head affixed to the first end of the handle;
c. the head comprised of an inner absorbent layer and an outer semi-permeable layer; and
d. a treatment liquid, the treatment liquid absorbed within the inner absorbent layer.

2. The applicator of claim 1, wherein the inner absorbent layer is a material selected from the group consisting of: cotton, wool, linen, hemp, and foam.

3. The applicator of claim 1, wherein the outer semi-permeable layer is a material selected from the group consisting of: cotton, microfiber, and silk.

4. The applicator of claim 1, wherein the treatment liquid is selected from the group consisting of: an antiseptic liquid, an antibiotic liquid, an anti-inflammatory liquid, and distilled water.

5. An applicator for cosmetic or medicinal use to provide treatment and relief to an area on a body, the applicator comprising:
a. a handle having a first end and a second end;
b. a head affixed to the first end of the handle, the head comprised of an inner layer and an outer layer;
c. the inner layer being an absorbent material;
d. the outer layer being a semi-permeable material;
e. a treatment liquid; and
f. the inner layer saturated with the treatment liquid;
g. whereby when the head contacts the body, the treatment liquid seeps out of the inner layer through the outer layer and onto the body.

6. The applicator of claim 5, wherein the absorbent material is selected from the group consisting of: cotton, wool, linen, hemp, and foam.

7. The applicator of claim 5, wherein the semi-permeable material is selected from the group consisting of: cotton, microfiber, and silk.

8. The applicator of claim 5, wherein the treatment liquid is selected from the group consisting of: an antiseptic agent, an antibiotic agent, an anti-inflammatory agent, and distilled water.

9. An individually packaged applicator for cosmetic or medicinal use, the applicator comprising:
a. a handle having a first end and a second end;
b. a head affixed to the first end of the handle; and
c. the head comprised of an inner layer and an outer layer;
d. whereby the head applies relief to an area on a person.

10. The applicator of claim 9, wherein:
a. the inner layer is an absorbent material; and
b. the outer layer is a semi-permeable material.

11. The applicator of claim 10, wherein the absorbent material is selected from the group consisting of: cotton, wool, linen, hemp, and foam.

12. The applicator of claim 10, wherein the semi-permeable material is selected from the group consisting of: cotton, microfiber and silk.

13. The applicator of claim 10, further comprising:
a. a treatment liquid;
b. wherein the treatment liquid is selected from the group consisting of: an antiseptic liquid, an antibiotic liquid, an anti-inflammatory liquid, and distilled water.

14. The applicator of claim 9, wherein:
a. the inner layer is a gel pack, the gel pack comprised of a gel encased within a plastic pouch; and
b. the outer layer is selected from the group consisting of: cotton, microfiber, silk, and plastic.

15. The applicator of claim 14, wherein the gel is selected from the group consisting of: ethylene glycol and diethylene glycol.
